(19) United States
(12) Patent Application Publication
(10) Pub. No.: US 2017/0290989 A1
(43) Pub. Date: Oct. 12, 2017

(54) CLIP SYSTEMS

(71) Applicant: Jonathan Mancini, Maple Ridge (CA)

(72) Inventor: Jonathan Mancini, Maple Ridge (CA)

(21) Appl. No.: 15/096,834

(22) Filed: Apr. 12, 2016

(51) Int. Cl. A61M 5/32; F16L 3/10; A61B 5/00; F16B 47/00; A61B 5/024; F16B 2/20; F16B 2/10

(52) U.S. Cl. CPC A61M 5/32; F16B 2/20; F16L 3/105; F16B 2/10; F16B 47/003; A61B 5/024; A61B 5/6838; A61M 2205/59

(57) ABSTRACT

A clip system relates to a sealed package having a picture or motivational item and a clip. The picture or motivational item has a double sided sticky adhesive with a protective layer. The protective layer is then removed and applied to a surface. The device can be applied to needles to help people overcome their fear. The device may also be applied to needles, heart monitors, walls and other surfaces. Various clips for holding medical instruments are also enabled.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of medical devices and more specifically relates to a clip system.

### 2. Description of Related Art

Spending day after day in bed or in the hospital can be a challenging nightmare. Whether the hospital stay is just a few weeks, months, or years. It can be isolating, boring, uncomfortable, scary, depressing, unhealthy, dependent and unproductive. Beeping machines, frequent needle sticks, unpredictable waits to see the doctor, unappetizing food and sleep deprivation are among the barrage of stressors that patients may experience in hospitals. Needles are one of the many instances under medical care, when a person is put into a potentially stressful and uncomfortable situation. A more comforting means to provide treatment to individuals that helps encourage them is desirable.

Several attempts have been made to solve the above-mentioned problems such as those found in U.S. Pub. Nos. 2008/0139076 to Karen Frances Frasier-Scott; 2005/0090783 to Wilmer Sibbitt; 2005/0209567 to Wilmer Sibbitt; 2012/0061286 to Maria Angelica Hueb De Menezes Oliveira, U.S. Pat. No. 3,299,891 to Smeton Robert L; U.S. Pat. No. 6,045,360 to Dionisio Rio Simoes, and International Patent No. JP2003275307 to Kaneda Toshio; KR100834541 to Ahn Jun Seo; CN204106717 to Cai Xiaoyun; CN204017127 to Zhang Lingyun; CN204033988 to Cattle Run Flowers in Burgundy Mei; and CA2562528 to Terrina Mingle. This art is representative of medical devices. However, none of the above inventions and patents, taken either singly or in combination, is seen to describe the invention as claimed.

Preferably, a clip system should provide functional stress relief and, yet would operate reliably and be manufactured at a modest expense. Thus, a need exists for a reliable clip system to avoid the above-mentioned problems.

### BRIEF SUMMARY OF THE INVENTION

In view of the foregoing disadvantages inherent in the known medical device art, the present invention provides a novel clip system. The general purpose of the present invention, which will be described subsequently in greater detail is to provide a simple and efficient way to help take some ease off patients while being in the hospital and receiving care.

A portion of the present invention relates to a sealed package consisting of a picture or motivational item and a clip. The picture or motivational item may have a double sided sticky adhesive with a protective layer. The protective layer is then removed and applied to a surface. The device can be applied to needles to help people overcome their fear. The device may also be applied to needles, heart monitors, walls and other surfaces.

A clip system of the present invention is also disclosed herein, in a preferred embodiment, comprising a clip assembly comprising a clip having a body comprising a back-plate and a biasing member. As such, the clip system comprises the clip assembly. The clip assembly comprises the clip. The clip comprises the body.

Referring now to the body; the body is defined by the back-plate and the biasing member. The back plate preferably comprises an adhesive pad for fastening to a surface. The biasing member comprises teeth such that the clip assembly is adjustable relative to the supported item. The biasing member comprises an arcuate profile suitable for supporting the supported item when used. The supported item is effectively held between the back-plate and the biasing member via biasing means. The supported item comprises a periphery in most instances. The supported item may comprise a lumen for example or a needle or the like. The supported item is structured and arranged to transport at least one fluid; the supported item generally comprises a hollow inner volume. The at least one fluid comprises liquid in most instances. The clip system may comprise a fusion allowing for 360 degrees rotation. The clip comprises a top plate for securing at least one motivational item for encouraging a patient when viewed. The clip system is structured and arranged such that the supported item is held in position along a vertical surface for ready use.

A kit is described herein including: at least one clip, at least one fastener (fastening means), at least one supported item, and at least one motivational item.

A method of using a clip system is also disclosed herein comprising the steps of: securing a plurality of clip assemblies to a surface, securing at least one supported item for use by a patient, motivating said patient via at least one motivational item to promote wellness, and removing the at least one supported item during non-use periods or for refilling.

The present invention holds significant improvements and serves as a clip system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, clip system, constructed and operative according to the teachings of the present invention.

FIG. 1 shows a perspective view illustrating a clip system during an ‘in-use’ condition showing a clip having a body according to an embodiment of the present invention.

FIG. 2 is a perspective view illustrating a clip assembly comprising a clip having the body comprising a back-plate and a biasing member according to an embodiment of the present invention of FIG. 1.

FIG. 3 is a perspective view illustrating the clip assembly according to an embodiment of the present invention of FIGS. 1-2.

FIG. 4 is a perspective view illustrating a kit including various clips according to an embodiment of the present invention of FIGS. 1-3.

FIG. 5 is a flowchart illustrating a method of use for the clip system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a medical device and more particularly to a clip system as used to reduce the stressfulness of receiving medical treatment.

Generally speaking, the clip system may further comprise a c-style clamp or clip attachment for a needle. The c-style clamp or clip attachment may comprise plastic or rubber in order to wrap around different sizes of needles. The c-style clamp may comprise a top plate for securing at least one motivational item for encouraging a patient when viewed. Adhesive may be included to stick to a photo or item to assist in covering up a needle for patients and kids. Methods of attachments may include wall mounts, adhesive, or VELCRO® to stick to various surfaces. VELCRO® may comprise adhesive backing suck that it is able to couple to a clip and mount the clip to various surfaces.

The device is a multi-purpose invention and may be used for many applications including attaching to a heart rate monitor, finger, needles, an intravenous, and any other medical products, toys, and decorations. Photo(s) or item(s) may be attached to various surfaces in a room of the patient as motivational means. Photo(s) or item(s) may include for example cartoons, animal, cars, poems, or family photos. C-style clamp or clip may comprise metal, plastic, rubber, or other suitable materials. Photo(s) or item(s) may comprise cardboard, plastic, laminated paper, paper, or other suitable materials. The device further comprises sanitized packaging which includes parchment paper, wax paper or other suitable materials. The clip system is designed to distract patients when receiving treatment.

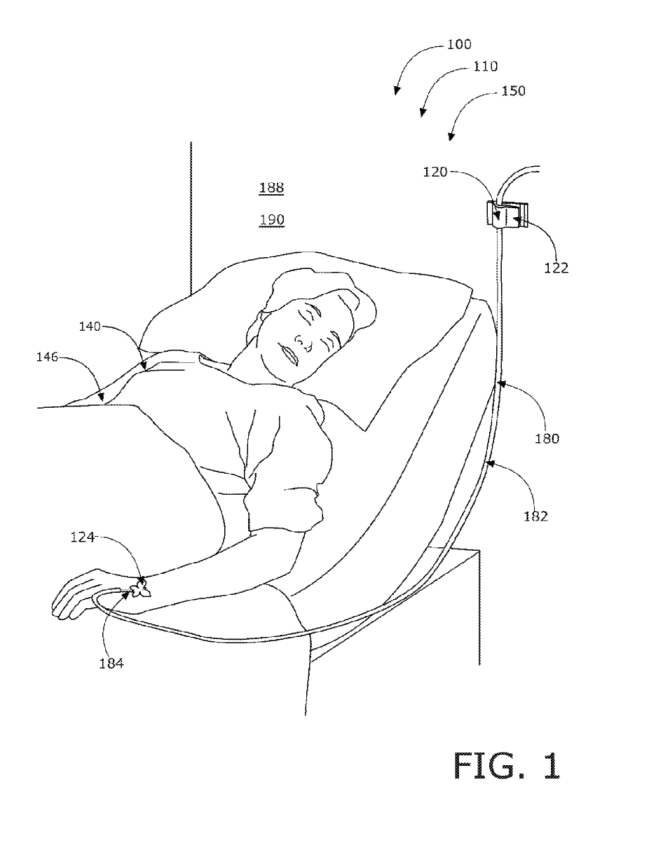

Referring to the drawings by numerals of reference there is shown in FIG. 1, clip system 100 having clip assembly 110 comprising clip 120 having body 130 comprising back-plate 132 and biasing member 152. Clip system 100 comprises clip assembly 110. Clip assembly 110 comprises clip 120. Clip 120 comprises said body 130. Body 130 is defined by back-plate 132 and biasing member 152. Back plate 132 may comprise adhesive pad 134 for fastening to surface 190. Other fastening means may be used. Supported item 180 is effectively held between back-plate 132 and biasing member 152. Supported item 180 comprises a periphery. Clip system 100 is structured and arranged such that supported item 180 is held in position along vertical surface 188. Biasing member 152 may comprise teeth 154 in certain embodiments such that clip assembly 110 is adjustable relative to supported item 180. Supported item 180 may for example comprise lumen 182.

Supported item 180 is structured and arranged to transport at least one fluid 192. Supported item 180 comprises a hollow inner volume. At least one fluid 192 may comprise air 194. At least one fluid 192 comprises liquid 196. Clip system 100 may comprise extrusion 170 allowing for 360 degrees rotation. Biasing member 152 comprises an arcuate profile suitable for supporting supported item 180 when used. Supported item 180 may alternately comprise needle 184.

L-shaped base 136 extrudes from front of back-plate 132. Biasing member 152 comprises a push-tab 156 for engaging and releasing biasing member 152. Back-plate further 132 comprises at least one aperture 142 for at least one fastener 144 to secure back plate 132 to at least one surface 190. At least one fastener 144 comprises bolts. Clip 120 comprises top plate 122 for securing at least one motivational item 124 for encouraging patient 146 when viewed. Biasing member 152 comprises c-style clamp 148.

Clip system 100 may be sold as kit 440 comprising the following parts: at least one clip at least one fastener; at least one supported item; and at least one motivational item, components as mentioned previously. The kit has instructions such that functional relationships are detailed in relation to the structure of the invention (such that the invention can be used, maintained, or the like in a preferred manner). Clip system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different decorative and securing combinations, parts may be sold separately, etc., may be sufficient.

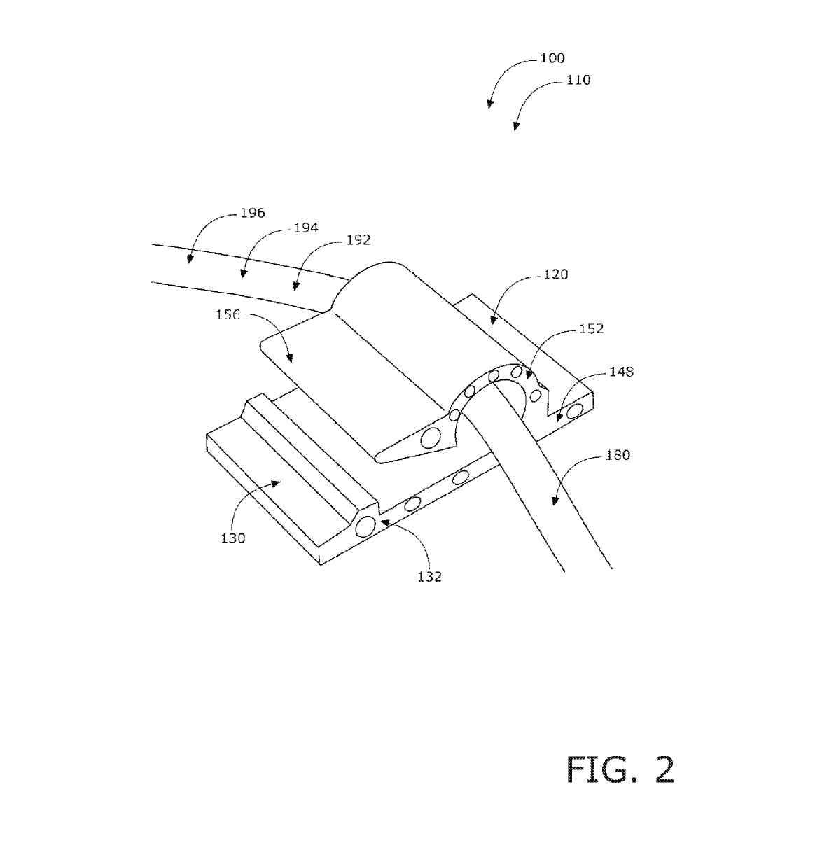

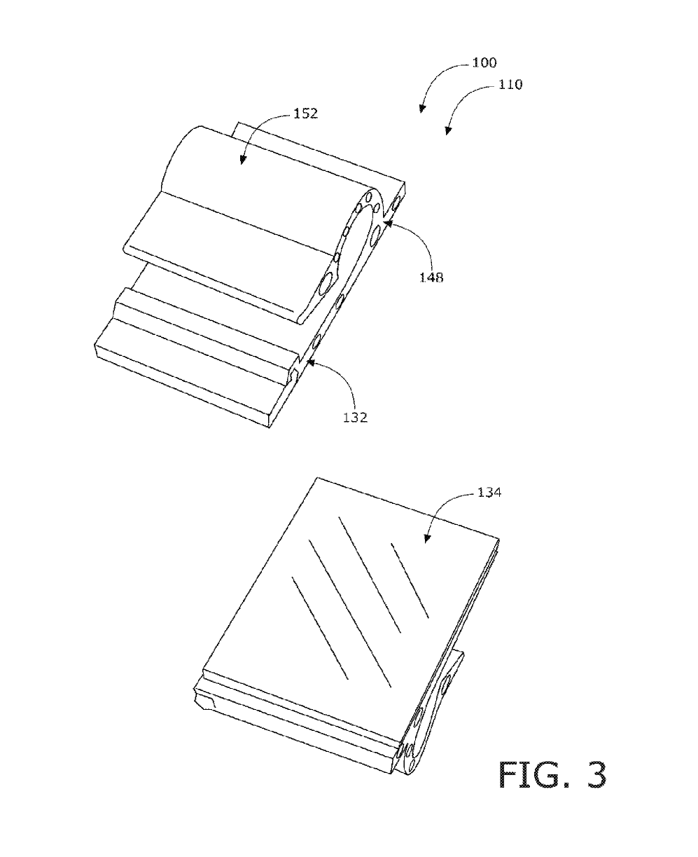

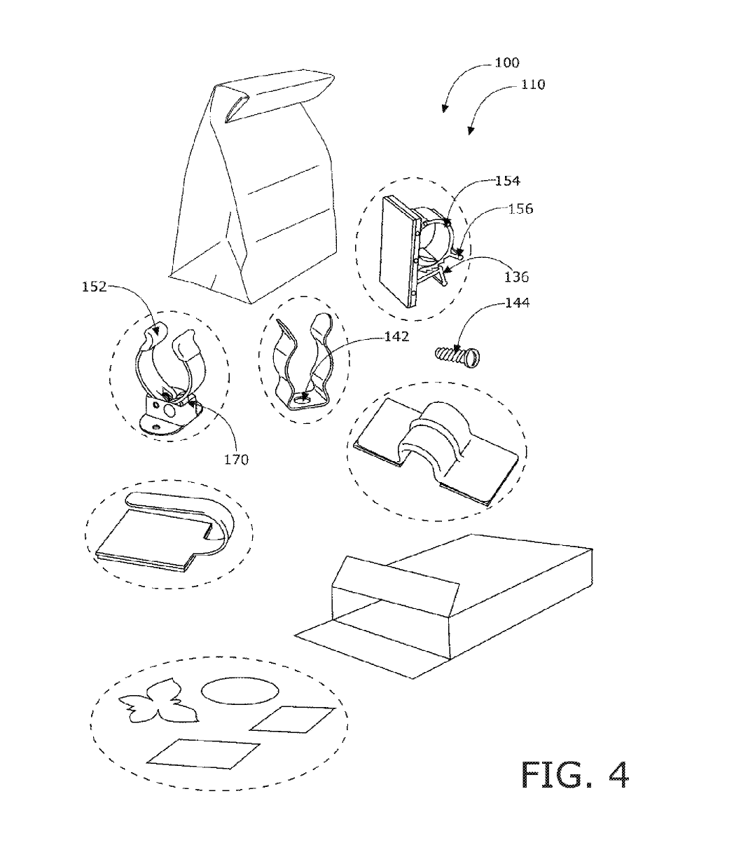

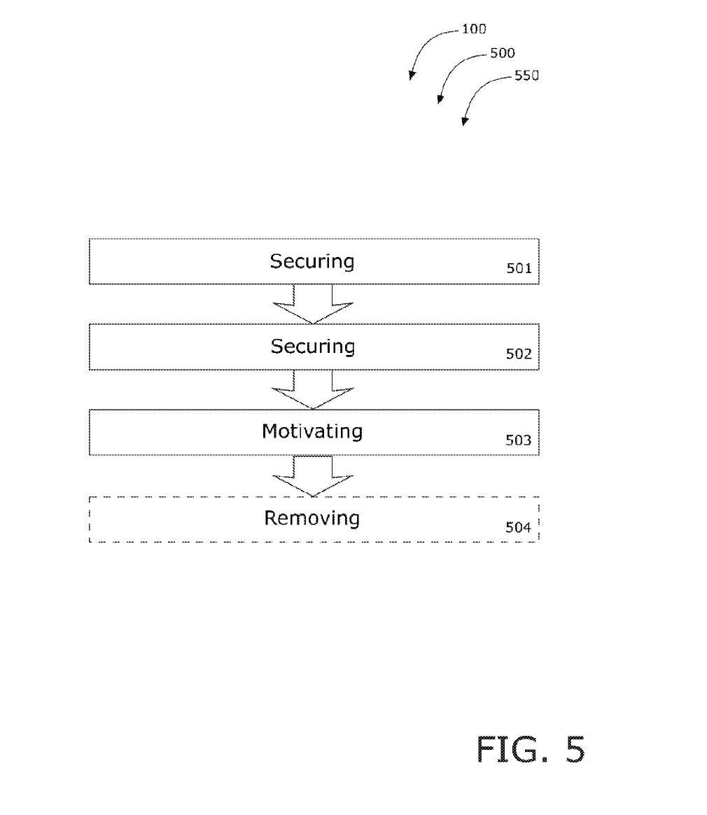

Referring now to FIG. 5 showing flowchart 550 illustrating method of use 500 for clip system 100 according to an embodiment of the present invention of FIGS. 1-4. As shown, method of use 500 may comprise the steps of: step one 501, securing a plurality of clip assemblies 110 to surface 190; step two 502, securing at least one supported item 180 for use by patient 146; step three 503, motivating patient 146 via at least one motivational item 124 to promote wellness; step four 504, removing at least one supported item 180.

It should be noted that step four 504 is an optional step and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A clip system comprising:
a clip assembly comprising;
a clip having;
a body comprising;
a back-plate; and
a biasing member;
wherein said clip system comprises said clip assembly;
wherein said clip assembly comprises said clip;
wherein said clip comprises said body;
wherein said body is defined by said back-plate and said biasing member;
wherein a supported item is effectively held between said back-plate and said biasing member;
wherein said supported item comprises a periphery; and
wherein said clip system is structured and arranged such that said supported item is held in position along a vertical surface for use.

2. The clip system of claim 1 wherein said biasing member comprises teeth such that said clip assembly is adjustable relative to said supported item.

3. The clip system of claim 2 wherein said supported item comprises a lumen.

4. The clip system of claim 1 wherein said back plate comprises an adhesive pad for fastening to a surface.

5. The clip system of claim 1 wherein said supported item is structured and arranged to transport at least one fluid, said supported item comprises a hollow inner volume.

6. The clip system of claim 5 wherein said at least one fluid comprises air.

7. The clip system of claim 5 wherein said at least one fluid comprises liquid.

8. The clip system of claim 1 wherein said clip system comprises an extrusion allowing for 360 degrees rotation.

9. The clip system of claim 1 wherein said biasing member comprises an arcuate profile suitable for supporting said supported item when used.

10. The clip system of claim 1 wherein said supported item comprises a needle.

11. The clip system of claim 2 wherein said a substantially L-shaped base extrudes from a front of said back-plate.

12. The clip system of claim 11 wherein said biasing member comprises a push-tab for engaging and releasing said biasing member.

13. The clip system of claim 1 wherein said back-plate comprises at least one aperture for at least one fastener to secure said back plate to at least one surface.

14. The clip system of claim 1 wherein said at least one fastener comprises bolts.

15. The clip system of claim 1 wherein said clip comprises a top plate for securing at least one motivational item for encouraging a patient when viewed.

16. The clip system of claim 1 wherein said biasing member comprises a c-style clamp.

17. A clip system comprising:
a clip assembly comprising;
a clip having;
a body comprising;
a back-plate; and
a biasing member;
wherein said clip system comprises said clip assembly;
wherein said clip assembly comprises said clip;
wherein said clip comprises said body;
wherein said body is defined by said back-plate and said biasing member;
wherein said back plate comprises an adhesive pad for fastening to a surface;
wherein said biasing member comprises teeth such that said clip assembly is adjustable relative to said supported item;
wherein said biasing member comprises an arcuate profile suitable for supporting said supported item when used;
wherein a supported item is effectively held between said back-plate and said biasing member;
wherein said supported item comprises a periphery;
wherein said supported item comprises a lumen;
wherein said supported item is structured and arranged to transport at least one fluid, said supported item comprises a hollow inner volume;
wherein said at least one fluid comprises liquid;
wherein said clip comprises a top plate for securing at least one motivational item for encouraging a patient when viewed; and
wherein said clip system is structured and arranged such that said supported item is held in position along a vertical surface.

18. The clip system of claim 17 further comprising a kit including:
said at least one said clip;
said at least one fastener;
said at least one said supported item; and
said at least one motivational item.

19. A method of using a clip system comprising the steps of:
securing a plurality of clip assemblies to a surface;
securing at least one supported item for use by a patient; and
motivating said patient via at least one motivational item to promote wellness.

20. The method of claim 19 further comprising the step of:
removing said at least one supported item.
